(19) United States
(12) Patent Application Publication
(10) Pub. No.: US 2020/0330659 A1
(43) Pub. Date: Oct. 22, 2020

(54) WOUND THERAPY DEVICE, SHELL FOR WOUND THERAPY DEVICE AND WOUND THERAPY METHOD

(71) Applicant: Aatru Medical, Inc., Cleveland, OH (US)

(72) Inventors: James WU, North Olmsted, OH (US); John BUAN, Maple Grove, MN (US); Richard L. MIDDAUGH, Rocky River, OH (US); Thomas E. LASH, Shaker Heights, OH (US); Timothy WOJCIECHOWSKI, Westlake, OH (US)

(21) Appl. No.: 16/956,457

(22) PCT Filed: Nov. 28, 2018
(86) PCT No.: PCT/US2018/062909

§ 371 Date: Jun. 19, 2020

Related U.S. Application Data
(60) Provisional application No. 62/609,731, filed on Dec. 22, 2017.

(51) Int. Cl. A61M 1/00; A61F 13/02

(52) U.S. Cl. CPC A61M 1/009; A61F 13/0223

(57) ABSTRACT

A pump shell for a wound therapy device includes a shell body having an interior cavity for accommodating a pump adapted to create a negative pressure condition upon actuation thereof. The shell body is configured to be affixed to one or both skin surrounding a tissue site or a skin contacting assembly or element to create a negative pressure condition at the tissue site over which the skin contacting assembly or element is disposed. The shell body also configured to maintain the interior cavity at a minimum specified volume while the pump creates the negative pressure condition.

### BACKGROUND

Negative pressure is a term used to describe a pressure that is below normal atmospheric pressure. Known topical negative pressure devices range from cumbersome wrinkle reducing suction apparatuses to wound therapies that include fluid-permeable wound cavity filling elements, covering dressings, reasonably air-tight means for sealing against the skin, and drainage tubes connecting the wound site and cavity filling element to the vacuum source via a fluid collection canister.

To enable a more prolonged application of topical negative pressure, powered systems, which include a vacuum generation source such as a pump, have been developed and many examples of such systems are used today for skin treatments and restorative purposes like the temporary removal of wrinkles. Many of these systems, however, are not convenient for users. Such known systems can be large, heavy, noisy, uncomfortable, and not simple for users to apply and initiate a controlled pressure condition. Such known systems also rely on an outside power or vacuum source to create topical negative pressure conditions.

Such tissue treatment, surgery, and other advanced technical interventions are becoming more common given the occurrence of both the aging population, as well as increasingly compromised patient populations. This trend looks set to continue. In wound care, for example, healthcare professionals are now more likely to encounter wounds with complex healing problems that are difficult to manage. Attempts have been made to produce more simple mechanical devices able to apply topical and negative pressure to a tissue site. It will be appreciated that such a medical device, due to its relative simplicity of design, would be expected to reduce material costs and assembly costs. For example, attempts have been made to use a hand-pump system for the application of topical negative pressure at a tissue site. However, such a system fails to enable easier application by the user, discreet use, and prolonged convenient application of topical negative pressure, and, in fact, re-evacuation is often necessary. These can be serious deficiencies, particularly as many such systems are ideally useable for prolonged periods, such as overnight.

### SUMMARY

According to one aspect, a shell for a wound therapy device includes a shell body having an interior cavity for accommodating a negative pressure condition. The shell body is configured to be affixed to one or both skin surrounding a tissue site or a skin contacting assembly or element to apply the negative pressure condition at the tissue site over which the skin contacting assembly or element is disposed. The shell body also configured to maintain the interior cavity at a minimum specified volume while accommodating the negative pressure condition.

According to another aspect, a method for applying a negative pressure to a tissue site includes covering the tissue site with a skin contacting element, affixing a pump housing element having a pump accommodated therein to one or both skin surrounding the tissue site or the skin contacting element, and actuating the pump to create a pressure condition at the tissue site. The pump housing element is configured to maintain the interior cavity at a minimum specified volume while the pump creates the negative pressure condition.

According to a further aspect, a wound therapy device includes a skin contacting assembly or element configured for covering a tissue site and configured to allow at least one of liquid and air to pass therethrough from the tissue site. The wound therapy device further includes a pump for creating a negative pressure condition at the tissue site upon actuation thereof and a pump shell having an interior cavity for accommodating the pump. The pump shell configured to be affixed to one or both skin surrounding the tissue site or the skin contacting assembly or element to create a negative pressure condition at a tissue site. The pump shell is also configured to maintain an interior cavity thereof at a minimum specified volume while the pump creates the negative pressure condition.

According to still another aspect, a pump shell for a wound therapy device includes a shell body having an interior cavity for accommodating a pump adapted to create a negative pressure condition upon actuation thereof. The shell body is configured to be affixed to one or both skin surrounding a tissue site or a skin contacting assembly or element to create a negative pressure condition at the tissue site over which the skin contacting assembly or element is disposed. The shell body also configured to maintain the interior cavity at a minimum specified volume while the pump creates the negative pressure condition.

### BRIEF DESCRIPTION OF THE DRAWINGS

FIG. 1 is a schematic cross-sectional view of a wound therapy device having a skin contacting element or assembly and a pump housing element or assembly, with the pump housing element or assembly shown in a pre-affixed state (i.e., before being affixed to the skin contacting element).

FIG. 2 is a schematic cross-sectional view similar to FIG. 1 but showing the pump housing element affixed to the skin contacting element and to skin surrounding a tissue site at which the wound therapy device is being used.

FIG. 3 is a schematic upper perspective view of a pump shell according to an exemplary embodiment.

FIG. 4 is a schematic underside perspective view of the pump shell of FIG. 3.

FIG. 5 is a schematic perspective cross-sectional view of the pump shell of FIGS. 3 and 4.

FIG. 6 is a schematic side elevation view of the pump shell of FIG. 3.

FIG. 7 is a schematic end elevation view of the pump shell of FIG. 3.

FIG. 8 is a schematic upper perspective view similar to FIG. 3 but showing the pump shell relative to a plane in a pre-negative pressure condition.

FIG. 9 is a schematic upper perspective view similar to FIG. 8 but showing the pump shell relative to the plane in the negative pressure condition.

FIG. 10 is a schematic upper perspective view similar to FIG. 3 but showing a pump shell according to an alternate exemplary embodiment.

FIG. 11 is a schematic exploded perspective view of a pump shell and a retaining ring according to another alternate exemplary embodiment.

FIG. 12 is a schematic cross-sectional view of the pump shell and retaining ring of FIG. 11 shown affixed to the skin and surrounding a skin contacting element or assembly.

FIG. 13 is a schematic cross-sectional view of a pump shell and a retaining ring according to yet another alternate exemplary embodiment.

FIG. 14 is a schematic upper perspective view of a pump shell having tapering according to an alternate exemplary embodiment.

### DETAILED DESCRIPTION

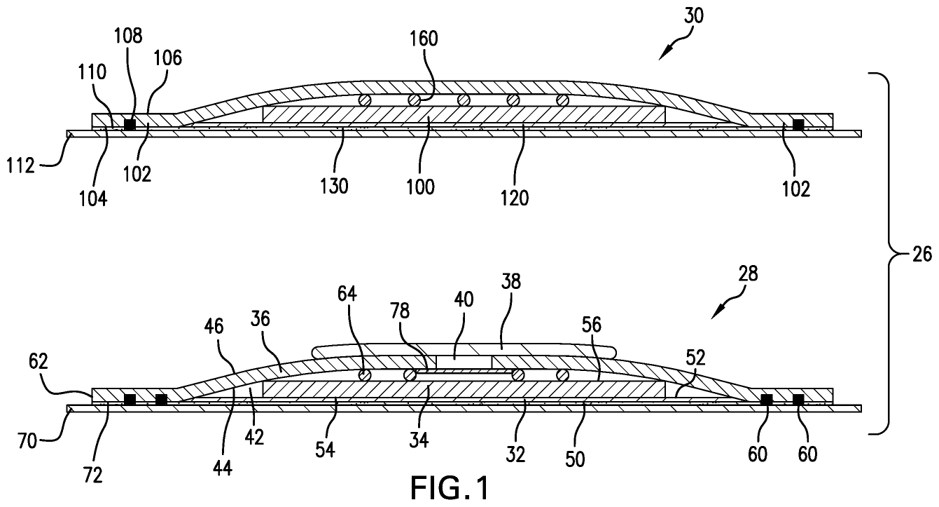

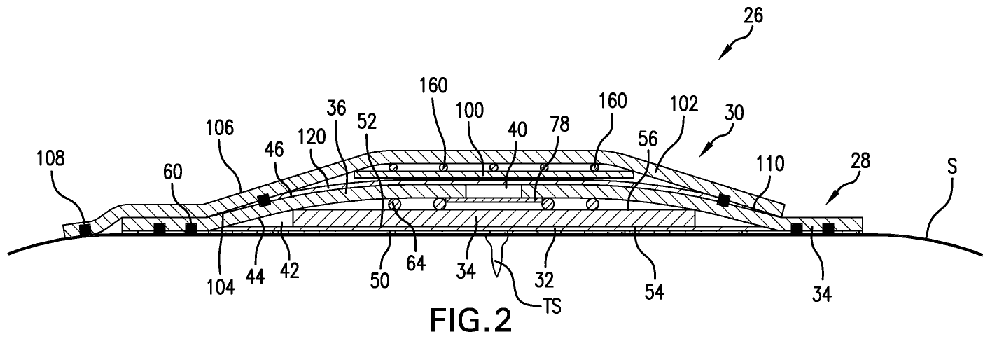

FIGS. 1 and 2 illustrate a known wound therapy device 26 including a skin contacting element or assembly 28 and a pump housing element or assembly 30 accommodating a pump 100 therein. In use, the skin contacting assembly 28 is positioned on and/or affixed to skin S surrounding a wound site or tissue site TS (FIG. 2). The pump assembly 30 can then be affixed to one or both the skin S surrounding the tissue site or the skin contacting assembly 28 to provide reduced pressure (typically below that of atmospheric pressure and alternatively referred to as negative pressure) to the tissue site TS.

As shown, the skin contacting assembly 28 can include a skin contact layer 32, a wicking element 34, a drape 36, an upper peel-away layer 38 and a release liner 70. The drape 36 covers the skin contact layer 32 and the wicking element 34. The drape 36 can include at least one opening 40 therethrough that provides fluid communication to an enclosed volume 42 defined under the drape 36 (i.e., between the drape 36 and the skin S). More particularly, the at least one opening 40 extends from a lower (inner) surface 44 of the drape 36 to an upper (outer) surface 46 of the drape 36. In one embodiment, the at least one opening 40 is a single elongated or slot opening extending longitudinally along a longitudinal extent of the drape 36. The peel-away layer 38, which can also be or be referred to as a casting sheet, removably covers the at least one opening 40. The peel-away layer 38 is releasable from the drape 36 when the pump housing assembly 30 is ready to be affixed to the skin contacting assembly 28. Similarly, the release liner 70 is releasable from the skin contacting side 50 when the skin contacting assembly 28 is ready to be positioned on and/or affixed to the skin S surrounding the tissue site TS.

The skin contact layer 32 (i.e., the bottom-most element of the skin contacting assembly 28) can be provided in a configuration that generally sticks or adheres to the skin S or can be provided in a configuration that generally does not stick or adhere to the skin S. More particularly, in one embodiment, the skin contact layer 32 can have adhesive properties and can be formed, for example as a perforated polyurethane film coated with silicone adhesive (or some other tissue or skin friendly adhesive). In another embodiment, the skin contact layer 32 can be provided without adhesive properties and/or can be provided with anti-stick properties. For example, the skin contact layer 32 could be formed as a silver coated nylon or some other known non-stick wound interface material. In these or other embodiments, the skin contact layer 32 can be made from an elastomeric material, such as a polymeric material that has rubber-like properties. The elastomeric material can be a thin, flexible elastomeric film.

In any of the foregoing embodiments, the skin contact layer 32 can include a plurality of openings (not shown) that allow exudate from the tissue site TS to pass through the skin contact layer 32. When used in conjunction with the wicking element 34, the exudate can pass through the skin contact layer 32 and be retained within the wicking element 34. As shown, the skin contact layer 32 includes a skin contacting side 50, which contacts the skin S and/or the tissue site TS when the skin contacting assembly 28 is placed over the tissue site TS (as shown in FIG. 2). The skin contact layer 32 also includes an upper side 52 opposite the skin contacting side 50 that faces away from the tissue site TS when the skin contacting assembly 28 is affixed to the skin S over the tissue site TS.

The wicking element 34 can be made from an absorbent material capable of absorbing liquid so as to absorb exudate from the tissue site TS. The wicking element 34 includes a skin facing side 54 that faces the skin S and the tissue site TS when the skin contacting assembly 28 is affixed to the skin S over the wound or tissue site TS. The wicking element 34 can also include an upper side 56 that is opposite the skin facing side 54 and faces away from the skin S when the skin contacting assembly 28 is affixed to the skin S over the tissue site TS. The wicking element 34 can be made from a super absorbent polymer and absorbent beads, foams or natural absorbent materials. In the same or an alternate embodiment, the wicking element 34 can include a pressure distribution layer (not shown) that has low density (i.e., significant airspace) three dimensional fabric that functions to distribute the negative pressure about the surface area of the skin contacting assembly. Optionally, the wicking element 34 can be configured with an aperture (not shown) defined therethrough, such as a slotted aperture.

The drape 36 covers the wicking element 34 and the skin contact layer 32. The drape 36 can be made from a flexible material and/or can be made from a thin, flexible elastomeric film. Examples of such materials include polyurethane or polyethylene films. The drape 36 can be configured to inhibit passage of air and liquid through the drape 36 other than through the opening 40. For example, the drape 36 can be made from a material that is air and liquid impermeable, or the drape 36 can be coated with a substance, e.g., a hydrogel or hydrocolloid, or can be metalized, to inhibit passage of air and liquid through the drape 36 other than through the opening 40. In alternative embodiments, the drape 36 can be made from semipermeable materials that can maintain moisture around the tissue site TS while being permeable to water vapor, oxygen, nitrogen and/or other gases.

The skin contacting assembly 28 further includes a sealing element 60 (e.g., two peripheral seals in the illustrated embodiment, though this is not required and could be a single peripheral seal or more than two peripheral seals in other embodiments). Since the drape 36 can be made from a flexible film-like material, small air passage ways can be formed between the skin S and the drape 36 when the drape 36 is affixed to the skin S around the tissue site TS. The sealing element 60 is positioned on the skin contacting side 50 of the skin contact layer 32, particularly on the lower surface 44 of the drape 36. The sealing element 60 is configured to preclude gas and liquid from passing through any channels formed between the skin contact layer 32 or the drape 36 and the skin S and exiting or entering around a peripheral edge 62 of the drape 36. The sealing element 60 is configured to preclude gas and liquid from passing between the skin S and the skin contact layer 32 (or the wicking element 34 if included) or the drape 36 when the drape 36 and the sealing element 60 are applied to the skin S. The sealing element 60 operates similar to a gasket and can be made from a hydrogel material, or any other material that can prevent the migration of air and liquid from the tissue site TS under the drape 36 or the skin contact layer 32 and over the skin S. The sealing element 60 is schematically depicted in FIGS. 1 and 2, and can be made to include multiple rings or deposited in a manner to provide a tortuous path through which air and liquid must pass between the skin S and the wound contact layer 32 (or the wicking element 34) or the drape 36 when the drape 36 and the sealing element 60 are applied to the skin S.

The skin covering assembly 28 can also include at least one spacer element 64, though this is not required. The spacer element 64 is configured to maintain spacing between the drape 36 and the tissue site TS covered by the drape 36. Since the drape 36 can be made from a flexible material, as reduced pressure is applied in the enclosed volume 42, the drape 36 may be drawn towards the skin S and the tissue site TS. In situations where the pump housing assembly 30 reacts with selected gases in the air to remove these selected gases from the enclosed volume 42 to reduce pressure, having the drape 36 collapse toward the tissue site TS can result in the pressure in the enclosed volume 42 increasing toward ambient, which is undesirable for negative pressure wound therapy. Spacer element 64 can be a frame structure or another structural element to provide volume control so as to maintain an appropriate spacing between the drape 36 and the tissue site TS when reduced pressure is being applied to the enclosed volume 42. Spacer element 64 could also be a flexible coil spring, which may allow for more flexibility of the skin contacting assembly 28 over the tissue site TS when the skin contacting assembly 28 is affixed to the skin S. The spacer element 64 can be configured to conform to curves found on the human body while maintaining adequate spacing between the drape 36 and the tissue site TS.

As mentioned above, the skin contacting assembly 28 can also include the release liner 70. The release liner 70 can be disposed over the skin contact side 50 of the drape 36. The release liner 70 is removable and can be provided to expose the adhesiveness of the skin contact layer 32. For example, when the skin contact layer 32 is formed so as to have adhesive properties, the release liner 70 is removable to expose such properties. When the skin contact layer 32 is formed without adhesive properties, an adhesive 72 can be provided on the skin contact layer 32, and particularly on the skin contacting side 50 of the skin contact layer 32, so that removal of the release line 70 exposes the adhesive 72. The release liner 70 is removable from the skin contact layer 32 prior to affixing the skin contacting assembly 28 to the skin S and over the tissue site TS.

The skin contacting assembly 28 can further include an air permeable liquid impervious membrane 78 that covers the opening 40 in the drape 36. In the illustrated embodiment, the air permeable liquid impervious membrane 78 is affixed to the lower surface 44 of the drape 36; however, the air permeable liquid impervious membrane 78 could also be disposed on the outer surface 46 of the drape 36 covering the opening 40 in the drape 36. In any case, the air permeable liquid impervious membrane 78 precludes liquid (e.g., exudate) from traveling from the wicking element 34 through the opening 40 toward the pump housing assembly 30 when the pump housing assembly 30 is affixed to the skin contacting assembly 28, such as shown in FIG. 2.

As mentioned above, the peel-away layer 38 is releasable from the drape 36 when the skin contacting assembly 28 is affixed to the skin S around the tissue site TS. With the peel-away layer 38 removed from the drape 36, the pump housing assembly 30 can be affixed to the skin contacting assembly 28 and/or to the skin S to provide negative pressure to the tissue site TS. The pump housing assembly 30 generally includes the pump 100 and a pump drape 102. The pump drape 102 is configured to affix to the drape 36 and/or the skin S around the tissue site TS and cover the opening 40 in the drape 36 after the peel-away layer 38 has been removed from the drape 36. FIG. 2 depicts the left side of the pump drape 102 affixed to the skin S and the right side affixed to the drape 36. The pump drape 102 could be made larger so that the pump drape 102 contacts the skin S and surrounds the peripheral edge 62 of the drape 36 (or could be disposed so as to not be offset relative to the drape 36). Also, the pump drape 102 could be made smaller so that the pump drape 102 is affixed only to the wound drape 36.

The pump drape 102 includes a lower side 104 and an exterior side 106 opposite the lower side 104. The lower side 104 of the pump drape 102 is the side of the pump drape 102 that contacts the outer surface 46 of the drape 36 when the pump housing assembly 30 is affixed to the skin contacting assembly 28 and/or that contacts the skin S when the pump drape 102 is affixed to the skin S. The exterior side 106 of the pump drape 102 is exposed to ambient in the illustrated embodiment.

The pump housing assembly 30 also includes a pump sealing element, which can include a pump gasket 108 and/or adhesive 110. The pump sealing element 108 and/or 110 can be positioned on the lower side 104 of the pump drape 102. The adhesive 110 can be an adhesive that is stronger or more aggressive than the adhesive 72 on the drape 36, for example when the adhesive 110 only comes in contact with the drape 36 and not the skin S. The pump gasket 108 can be made from the same material, e.g., a hydrogel, and operate similarly to the sealing element 60 described hereinabove. The pump gasket 108 can contact the skin S, for example when the pump drape 102 is larger than the skin contacting assembly 28 and/or offset relative to the skin contacting assembly. The pump sealing element 108, 110 can be configured to preclude ingress of air between the pump drape 102 and the drape 36 when the pump drape 102 is affixed to the drape 36 and/or to preclude ingress of air between the pump drape 102 and the skin S when the pump drape 102 is affixed to the skin S.

The pump housing assembly 30 also includes a release liner 112 that covers the pump sealing element, which can be the pump gasket and/or the adhesive 110. The release liner 112 is removed from the pump drape 102 prior to affixing the pump housing assembly 30 to the skin contacting assembly 28 and/or the skin S.

The pump 100 in the pump housing assembly 30 can be a pump configured to react with a selected gas found in air, a zinc/air cell, a mechanical pump, or another small pumping device that can provide reduced pressure to the enclosed volume 42 through the opening 40 when the pump housing assembly 30 is affixed to the skin contacting assembly 28. In an embodiment where the pump 100 is a reactor configured to react with a selected gas founded air, the pump 100 consumes the selected gas in the enclosed volume 42. In the embodiment where the pump 100 is such a reactor, the pump drape 102 covers the pump 100. An example of a reactor that can be used in the pump housing assembly 30 is described in US2014/0109890A1. US2014/0109890A1 describes an oxygen based heater; however, the oxygen based heater can be used as the reactor to consume oxygen within the enclosed volume 42 thus producing a partial vacuum within the enclosed volume 42 (i.e., a negative pressure condition). The reactor can include a reducing agent, a binding agent on a reactor substrate, and an electrolyte solution, which can be provided in an electrolyte impregnated pad. The reducing agent on the reactor substrate can be zinc, aluminum, or iron, for example.

The pump housing assembly 30 can further include an air permeable liquid impervious membrane 120, which can be similar to the air permeable liquid impervious membrane 78 that covers the opening 40 in the wound drape 36. In the embodiment where the pump 100 is a pump that consumes oxygen in the enclosed volume 42, the pump 100 is interposed between the air permeable liquid impervious membrane 120 and the pump drape 102 when the pump drape 102 is affixed to the wound drape 36 covering the opening 40 in the wound drape 36. In another embodiment, not shown, the air permeable liquid impervious membrane 120 can also envelope the pump 100.

The pump housing assembly 30 can also include a removable seal layer 130 that prevents the pump 100 from being exposed to ambient oxygen until after removal of the removable seal layer 130. In the embodiment where the pump 100 is a reactor configured to react with oxygen, both the pump drape release liner 112 and the removal seal layer 130 are removed from the pump housing assembly 30 prior to affixing the pump housing assembly 30 to the skin contacting assembly 28 and/or to the skin S. If desired, the pump drape release liner 112 can be attached to the removable seal layer 130 so that removal of the pump drape release liner 112 from the pump drape 102 results in removal of the removable air seal 130 exposing the pump 100 to ambient. In another alternative arrangement, the pump drape release liner 112 can be affixed to the pump drape 102 in a manner to prevent the pump 100, which in this embodiment would be a reactor configured to consume oxygen, from being exposed to ambient until after removal of only the pump drape release liner 112, e.g., the removable seal layer 130 may not be provided. In alternate embodiments, the pump 100 can be a zinc/air cell that reacts with oxygen found in the enclosed volume 42.

Alternatively, in lieu of the reactor and the zinc/air cell described herein (or in addition thereto), the pump 100 may be one of any combination of electro-chemical pumps, vacuum-on-demand devices (referred to herein as VOD), electrolyzers, pressure-reducing solid state devices, oxygen absorbing iron packets, or getters of zirconium titanium, vanadium iron, lithium, lithium metal, magnesium, calcium, lithium barium combinations, zinc-air battery, zinc-air battery components or other materials highly reactive with the selected gases, for example, nitrogen, carbon dioxide and oxygen gases found in wound bed environments. Further, each of the skin contacting assembly 28, the pump assembly 30 and the pump 100 can be any of the corresponding skin contacting assemblies, pump assemblies 30 and/or pumps 100 disclosed in either co-owned U.S. patent application Ser. No. 15/478,327 filed on Apr. 4, 2017 or co-owned PCT Patent Application No. PCT/US2016/059364 filed on Oct. 28, 2016, both disclosures expressly incorporated herein in their entireties. In one embodiment, the pump 100 can be a chemical pump and the shell body 202 can be designed to withstand negative pressure from about −10 mmHg to −175 mmHg.

As shown, the pump assembly 30 of the illustrated embodiment can further include at least one spacer element 160 covered by the pump drape 102. The spacer element 160 in the pump assembly 30 can be similar in configuration and function to the spacer element 64 provided in the skin contacting assembly 28. The spacer element 160 in the pump assembly 30 is configured to maintain spacing between the pump drape 102 and the wound drape 36 when reduced pressure is applied under the wound drape 36, i.e., within the enclosed volume 42, or when flexing on the skin S can alter the internal volume 42.

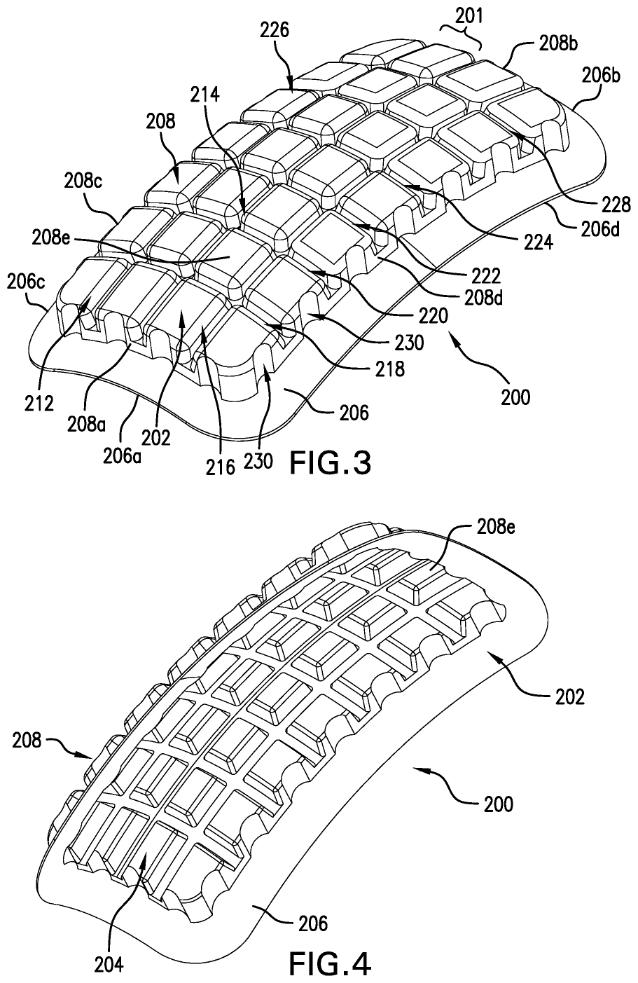

With reference now to FIGS. 3-5, a shell 200 for a wound therapy device is illustrated according to an alternate exemplary embodiment. As shown, the shell 200 includes a shell body 202 having or defining an interior cavity 204. In one application, the shell body 202 and the interior cavity 204 are configured for accommodating a negative pressure condition therein. In the same or another embodiment, the shell body 202 and the interior cavity 204 can be configured and/or sized for accommodating a pump (e.g., pump 100) adapted to create the negative pressure condition within the interior cavity 204 upon actuation thereof. Accordingly, the shell 200 can be provided with the pump 100 as described hereinabove regarding the pump 100 being provisioned with the pump housing assembly 30 of FIGS. 1 and 2. Alternatively, the negative pressure condition can be created without the use of a pump accommodated within the shell body 202, optionally including without the use of any pump at all.

In these or other embodiments, the shell body 202 can be configured to be affixed to a skin contacting assembly or element (e.g., the skin contacting assembly 28) and/or to the skin S surrounding the tissue site TS. In one embodiment, the shell body 202 is configured to maintain the interior cavity 204 at a minimum specified volume for at least a specified period of time while a negative pressure condition is present within the shell body (e.g., while pump 100 creates the negative pressure condition or while a negative pressure condition is otherwise provided within the shell body 202). For example, the shell body 202 can maintain an interior volume while the negative pressure condition is present that is no more than 19% less than its original interior volume (i.e., the volume of the shell body 202 before any negative pressure condition exists). In a more specific example, the shell body 202 can maintain an interior volume while the negative pressure condition is present that is no more than 10% less than its original volume. Optionally, the shell 200 of FIGS. 3-5 can be substituted for the pump drape 102 in the embodiment of FIGS. 1-2.

In any application, advantageously, the shape of the shell 200, and particularly the shell body 202 thereof, can be maintained by the features provided therein and thus the shell 200 is able to maintain the interior cavity 204 at the minimum specified volume while a negative pressure condition occurs within the shell 200. For example, the negative pressure condition can be created by the pump 100 when the shell 200 is used with the pump 100. As compared to the pump drape 102 of FIGS. 1-2, the shell 200 can be used without spacer elements 160 as the features of the shell 200 sufficiently maintain its shape and/or sufficiently maintain the interior cavity 204 at the minimum specified volume while a negative pressure condition is exhibited within the shell 200. Also, advantageously, the shell 200 enables negative pressure to occur without the need for a cumbersome external air pump attached to a hose, which is burdensome to the person being treated.

In one embodiment, as will be described in further detail below, the shell 200, and particularly the shell body 202 thereof, includes a plurality of geometric structures or structural portions 201. These can function to maintain a desired shape for the shell body 202 and/or to maintain the interior cavity 204 at the minimum specified volume during the negative pressure condition. In one embodiment, each of the geometric structures 201 can function to maintain about 155 mmHg (3 psi) of pressure without any volume loss from within the geometric structure 201. In the same or another embodiment, the shell body 202 having the geometric structures 201 can shrink in volume no more than about 19% when a negative pressure condition (e.g., 50-150 mmHg) occurs within the shell 200.

In one embodiment, the shell body 202 can include or be used with the air permeable liquid impervious membrane 120 arranged on an exposure side of the pump 100 so as to be interposed between the pump and the skin contacting assembly or element 28 when the shell body 202 is affixed to the skin contacting assembly or element or to the skin S. Further, the wicking element 34 can be interposed between the pump 100 and the tissue site TS such that the negative pressure condition created by the pump 100 in the shell 200 is applied to the tissue site TS through the wicking element 34.

In one embodiment, the negative pressure condition is 50-150 mmHg and thus the shell body 202 is configured to maintain the interior cavity 204 at a minimum specified volume while the pump creates a 50-150 mmHg negative pressure condition within the interior cavity 204. In the same or another embodiment, the shell body 202 is configured to maintain the interior cavity 204 at the minimum specified volume while the pump creates the negative pressure condition for at least 48 hours. By way of example, in these embodiments, the interior cavity 204 can have a volume of approximately 100 cubic centimeters and this volume can decrease no more than a specified amount (e.g., 19%) when the negative pressure condition occurs within the interior cavity 204.

Also by way of example, the shell body 202 can be formed from a polymer material, such as silicone rubber for example. In one embodiment, such silicone rubber can have a Shore hardness in the range of about 15A to about 90A. In a specific embodiment, the silicone rubber selected for the shell body 202 can have a Shore harness of around or about 20A. In one embodiment, the shell body 202 is formed from a material with a sufficiently high yield stress that is not pushed beyond its elastic limit when a 50-150 mmHg negative pressure condition occurs within the interior cavity 204. In these or other embodiments, the material selected for the shell body 202 and the Shore hardness of that material can correspond to a desired resiliency or rigidity of the shell body 202. In this manner, the shell body 202 and the interior cavity 204 defined thereby can shrink a desired minimum based, at least in part, on the selection of the material hardness for the shell body 202. Advantageously, when formed of silicone rubber, the shell body 202 can be oxygen permeable so as to allow sufficient oxygen to permeate through the shell body 202 to continue feeding the pump 100, particularly when the pump 100 is fed by oxygen. Alternatively, the shell body 202 could be permeable for another gas to pass or permeate through the shell body in sufficient quantities to feed the pump 100 when the pump 100 is configured to consume this other gas. In one embodiment, the shell body 202 can be formed via injection molding or a thermoforming process.

In these or other embodiments, portions of the shell body 202 can be co-molded such that at least one portion of the shell body 202 is formed of a first material and at least another portion of the shell body 202 is formed of a second material having a different Shore hardness relative to the first material, which maintaining the shell body 202 as an integrally formed (e.g., integrally molded) part). In a particular embodiment, the raised portion 208 can be formed of a suitable material that provides 155 mmHg (3 psi) of strength and the apron 206 can be formed of a more flexible material. In another particular embodiment, the geometric structures 201 are formed of a suitable material that provides 155 mmHg (3 psi) of strength and at one of connection portions of the raised portion 208 between the geometric structures and/or the apron 206 are formed of a different, more flexible material.

As shown, in the illustrated embodiment, the shell body 202 includes a perimeter apron 206 and a raised portion 208 extending upward from the perimeter apron 206 to define the interior cavity 204. To facilitate the shell body 202 maintaining the interior cavity 204 at a minimum specified volume while a pump contained therein creates the negative pressure condition, the raised portion 208 can comprise the plurality of geometric structures 201. In particular, in the illustrated embodiment, the raised portion 208 via the plurality of geometric structures 201 has a waffle shape, such as the waffle shape illustrated in FIGS. 3-5.

In the illustrated embodiment, the raised portion 208 has a generally rectangular configuration including longitudinal sides or ends 208a, 208b and lateral sides or ends 208c, 208d (sides 208a-208d are also alternatively referred to herein as walls). As shown, the apron 206 likewise has a generally rectangular configuration in the illustrated embodiment. Also as shown, the raised portion 208 is generally oriented so that the sides 208a-208d are generally parallel or extend along corresponding edges 206a-206d of the apron 206. In alternate embodiments, not shown, the raised portion 208 can be suitably shaped to adapt and/or complement various common shapes found on the human body.

In one embodiment, not shown, the geometric structures arranged about a perimeter of the raised portion 208 (i.e., those nearest the apron 206) can be contoured and/or reduced in height relative to more central ones of the geometric structures 201. In another embodiment, the raised portion 208 can be angularly arranged relative to the apron 206. As one non-limiting example, the raised portion 208 could be arranged at approximately forty-five degrees relative to the apron 206 so that the sides 208a-208d are angled at approximately forty-five degrees relative to the edges 206a-206d. In these or other embodiments, the size and shape of the raised portion 208 can vary and can be particularly adapted for fitting to a specific location on the human body (e.g., at the sternum) and/or to match contours at such a specific location. In the illustrated embodiment, the walls 208a-208d are each generally linearly arranged (i.e., formed in a straight line); however, it is to be appreciated that this is not required and the walls 208a-208d could each have some other configuration (e.g., one or more of the walls 208a-208d could be convex or concave).

Additionally, in the illustrated embodiment, the raised portion 208 includes at least one longitudinal groove (e.g., longitudinal grooves 212, 214 and 216) extending longitudinally from first longitudinal end 208a of the raised portion 208 to second, opposite longitudinal end 208b of the raised portion 208. The raised portion 208 of the illustrated embodiment also includes at least one lateral groove (e.g., lateral grooves 218, 220, 222, 224, 226 and 228) extending laterally from first lateral side 208c of the raised portion 208 to second, opposite lateral side 208d of the raised portion 208. In the illustrated embodiment, the at least one longitudinal groove includes three longitudinal grooves 212, 214 and 216 and the at least one lateral groove includes six lateral grooves 218, 220, 222, 224, 226 and 228. These grooves 212-228 together define the geometric structures 201 of the raised portion. It is to be understood and appreciated by those skilled in the art that the longitudinal grooves could include less than three or more than three longitudinal grooves. Likewise, the lateral grooves could include less than six or more than six lateral grooves. Also, it is to be appreciated that in alternate embodiments the shell body 202, and particularly the raised portion 208 thereof, can include only one or more longitudinal grooves or only one or more lateral grooves. In still a further alternate embodiment, the raised portion 208 could be provided without any grooves (i.e., provided in a single housing configuration).

As shown, in one embodiment, the at least one longitudinal groove and the at least one lateral groove (i.e., longitudinal grooves 212-216 and lateral grooves 218-228) can extend downward from an upper wall 208e of the raised portion 208 a first distance D1 that is less than a second distance D2 extending from the upper wall 208e to the perimeter apron 206. In one embodiment, not shown, the wall thickness of the raised portion 208 is thinner at the location of the grooves 212-228 than at adjacent portions defining the geometric structures. In still another embodiment, the portions of the raised portion 208 forming the grooves 212-228 can be formed of a different material, though integrally formed, as that which forms the geometric structures 201. For example, a first material having a lower Shore hardness can be used at the location of the grooves 212-228 and a second material having a higher Shore hardness (e.g., 70A) can be used for the geometric structures 201. This can provide increased flexibility between the geometric structures 201 to better enable the shell body 202 to conform to the human body when so applied.

As shown, in the illustrated embodiment, the raised portion 208 includes the upper wall 208e and at least one peripheral wall (e.g., peripheral walls defining the ends 208a, 208b, 208c and 208d) extending from the perimeter apron 206 to the upper wall 208e. In the illustrated embodiment, the at least one peripheral wall includes spaced apart embossments 230 that increase rigidity of the at least one peripheral wall and thereby increase rigidity of the shell body 202 to enable the shell body 202 to maintain the interior cavity 204 at the minimum specified volume while the pump contained therein creates the negative pressure condition. In one embodiment, the at least one longitudinal groove includes at least two longitudinal grooves (with three shown in the illustrated embodiment), the at least one lateral groove includes at least two lateral grooves (with six shown in the illustrated embodiment), and the spaced apart embossments include one embossment each positioned between adjacent ones of the at least two longitudinal grooves and the at least two lateral grooves. In the illustrated embodiment each embossment 230 extends from the perimeter apron 206 to the upper wall 208e.

By way of example only, in one embodiment, the interior longitudinal dimension from the first longitudinal side 108a to the second longitudinal side 108b can be about 7.5 inches (19.05 cm) and the interior lateral dimension from the first lateral side 108c to the second lateral side 108d can be about 3.5 inches (8.89 cm). In this same embodiment, the apron 206 can extend outwardly away from the raised portion 208 a distance of approximately 0.6 inches (1.524 cm) and the raised portion 208 can extend upward a distance of approximately 0.5 inches (1.270 cm). Also in the same embodiment, the corners of the apron 206 can have a radius of 0.50 inches (1.270 cm) and the corners of the raised portion 208 can have a radius of 0.35 inches (0.889 cm). In any of the embodiments, the thickness of the shell body 202 could be 0.2 cm, for example, and the interior cavity 204 can have a volume of about 100 cubic centimeters prior to any negative pressure condition occurring within the interior cavity 204. In one embodiment, the lateral width of the apron 206 can be within the range of about 0.25-1.00 relative to a lateral width of the raised portion 208.

Optionally, though not shown, the apron 206 can extend inward from the raised portion 208 to define an aperture that is smaller than an interior dimension of the raised portion 208. By way of example, the apron 206 could extend inwardly from the raised portion 208 about 1 cm so that the aperture defined by the apron 206 would be about 6.5 inches (17.399 cm) in the longitudinal direction and 2.5 inches (6.35 cm) in the lateral dimension. Of course, these and all dimensions mentioned herein are exemplary only and are not required. In one embodiment, the apron 206 can be formed integrally with the upper portion 208 but of a different material and/or with a reduced thickness. For example, the apron 206 can be formed of material having a lower Shore hardness than the upper portion 208 to provide more flexibility to the apron for complementing the contours of the human body on which the shell body 202 is applied.

In one embodiment, not shown, the apron 206 can include indentations (e.g., extending radially or peripherally from the raised portion to the edges 206a-206d) to add to flexibility of the apron 206 to allow it to better conform to the human body without undue wrinkling thereby reducing any vacuum loss from the interior cavity 204. Optionally, such indentations can be aligned with and/or extend from the grooves 212-228. Also, optionally, the shell body 202 can include a heat generating nano-needle silver film on an underside thereof to provide heat at the tissue site TS.

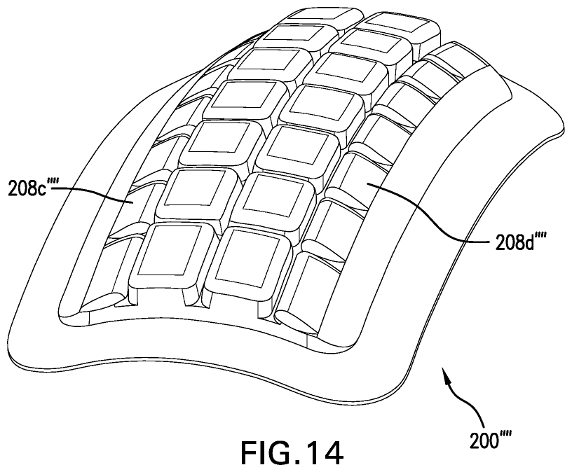

Also optionally, the raised portion 208 can include tapering. For example, the raised portion 208 can be tapered at least one of the longitudinal ends 208a, 208b, the lateral ends 208c, 208d and/or the corners defined at intersections of the ends 208a-208d. More particularly, the longitudinal ends 208a, 208b can include tapering, such as gradual tapering from a maximum height dimension of the raised portion 208 to the apron 206 or to an elevated height between the maximum height and the apron 206. Likewise, in addition or in the alternative, the lateral ends 208c, 208d can include tapering, such as gradual tapering from the maximum height dimension of the raised portion 208 to the apron 206 or to an elevated height between the maximum height and the apron 206. In addition to either or both of these, or in the alternative, the corners defined at intersections of the ends 208a-208d can include tapering, such as gradual tapering from the maximum height dimension of the raised portion 208 to the apron 206 or to an elevated height between the maximum height and the apron 206. Such tapering can advantageously reduce the likelihood of the raised portion 208 (and the shell 200 and any skin contacting assembly 28 thereunder) catching or snagging on external objects when worn by a person (e.g., the wearer's cloths, other hospital apparatus, such as hoses, beds, etc.). With reference to FIG. 14, one example of a tapered shell 200″″ is shown with only lateral ends 208c″″ and 206d″″ tapered.

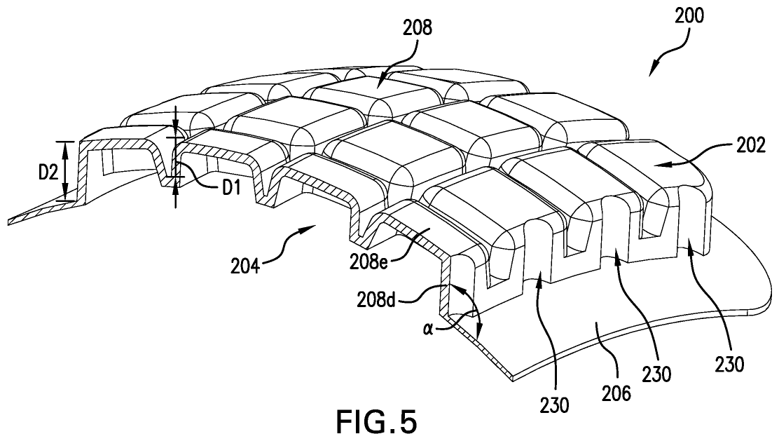

In the illustrated embodiment, as best shown in FIG. 5, an angle a between the longitudinal side 208d and the apron 206 can be an obtuse angle. This same angle αcan be provided between, respectively, each of the sides 208a-208c and the apron 206. For example, the angle α can be greater than 90 degrees, preferably in the range of about 95 degrees to 135 degrees, and more preferably in the range of about 115-125 degrees. In a particular example, the angle αis 121 degrees. In an alternate embodiment, not shown, the angle angel αcan be an acute angle (e.g., 90 degrees). Such an angle can inhibit lifting of the apron 206 from the skin S when applied and the negative pressure condition is created within the shell 200.

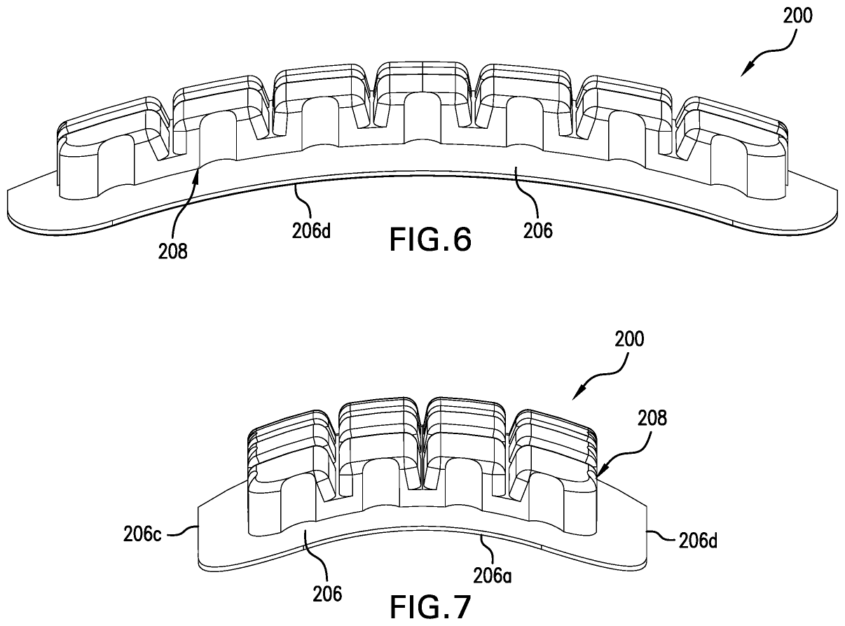

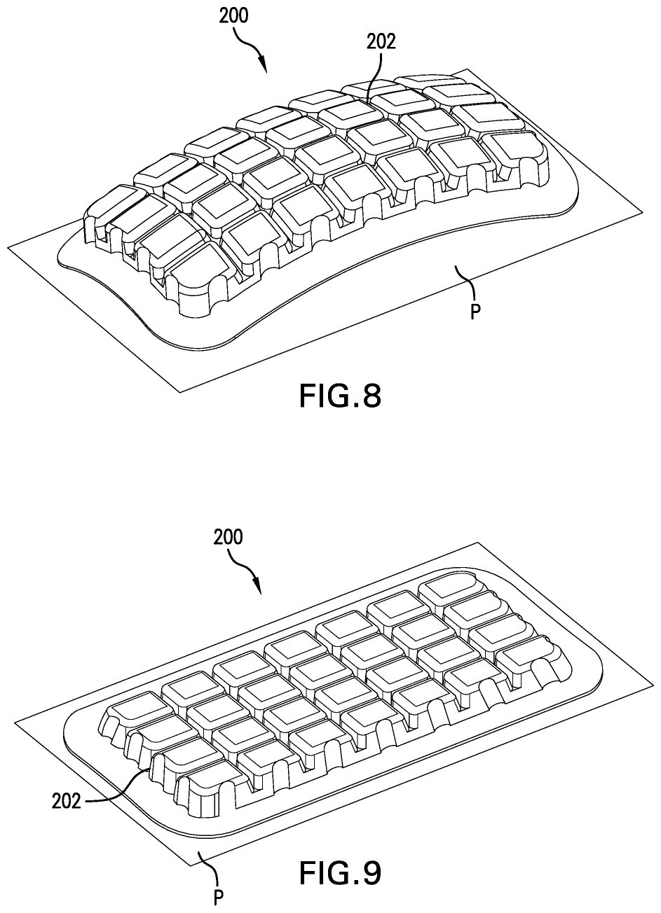

With reference to FIGS. 6 and 7, the shell 200, and particularly the shell body 202, can have longitudinal curvature and a lateral curvature. These can enable the shell body 202 to more easily conform to the curved contours of the human body. By way of example, the longitudinal curvature can have a curvature radius of about 14.0 cm (FIG. 6) and the lateral curvature can have a curvature radius of about 5.5 cm (FIG. 7), though other radii of curvature could be used. In particular, the longitudinal curvature of about 14.0 cm is shown by the edge 206d of the apron in FIG. 6 and the lateral curvature of about 5.5 cm is shown by the edge 206a in FIG. 7. Also, the longitudinal and lateral curvatures can function to provide resistance to the apron 206 and inhibit the apron 206 from bending upward with the interior cavity 204 is under negative pressure. With reference to FIGS. 8 and 9, the shell 200 is shown relative to plane P. In a pre-negative pressure state (i.e., before negative pressure is applied) shown in FIG. 8, the curvatures of the shell body 202 result in the shell body being raised at longitudinal and lateral central areas relative to the plane P. After the negative pressure condition shown in FIG. 9, the apron 206 abuts and rests flatly against the plane P.

While note shown, it is to be appreciated that the apron 206 and/or the raised portion 208 can have alternate shapes or configurations. For example, one or both the apron 206 and the raised portion 208 can be shaped to complementarily match contours of the body or body portion on which the shell body is intended for use. In one particular example, the apron 206 can have a butterfly configuration. Of course, as will be appreciated by those skilled in the art, other shapes or configurations can be used that correspond to particular body shapes (e.g., sacrum, axilla, elbow, etc.).

In an alternate embodiment, not shown, the shell body 202 can include a pressure release valve. The pressure release valve can be the same or similar to the pressure relief valve shown in co-owned PCT Patent Application No. PCT/US2016/059364 filed on Oct. 28, 2016, expressly incorporated herein by reference. The pressure relief valve can function to control pressure within the interior cavity 204 and/or the enclosed volume 42 of the skin contacting assembly 28. In addition or in the alternative, the pressure relief valve can allow for the reintroduction of atmospheric gases to restart the pump 100. More particularly, the pressure release valve can allow for selected communication between interior cavity 204 and/or the enclosed volume 42 and ambient. The pressure release valve can be operated when a predetermined pressure differential exists between the interior cavity 204 and/or the enclosed volume 42 and ambient. Alternatively, the shell body 202 could be purposefully punctured to instantly enable communication between the interior cavity 204 and/or the enclosed volume 42 with ambient. Optionally, the shell body 202 can be configured to enable such puncturing. For example, the shell body 202 can include a weakened area (e.g., an area with reduced wall thickness), optionally marked with indicia, for facilitating such puncturing.

In an alternate embodiment, the shell body 202 can be pre-compressed when the shell body 202 is sealed (e.g., to the skin contacting assembly 28 and/or to skin S) and subsequently released from compression to create a negative pressure force within the shell body 202. In a particular embodiment, the geometric structures 201 of the shell body 202 can be pre-compressed when the shell body 202 is sealed (e.g., to the skin contacting assembly 28 and/or to skin S) and subsequently released from compression to create a negative pressure force within the shell body 202. This can be supplemental to any negative pressure force created by the pump 100 within the shell body 202. Alternatively, this can be in substitution for negative compression created by any pump and thus with pre-compression the shell body 202 can be used without a pump. In these embodiments, the geometric structures 201 can be formed of a resilient material so that the pre-compression can be applied and, when the compression force removed, the resiliency of the material urges the geometric structures 201 to their uncompressed states while simultaneously created a negative pressure within the shell body 202. Optionally, the geometric structures 202 can be pre-compressed after the shell body 202 is sealed when used in combination with a pressure relief valve as describe hereinabove to enable the shell body to apply a desired negative pressure therein.

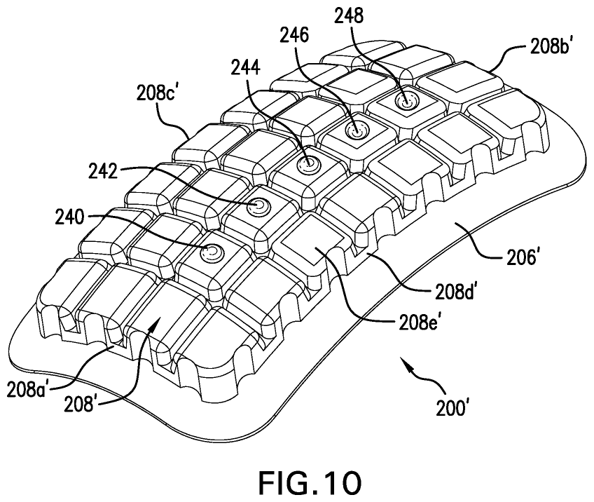

With reference to FIG. 10, a shell 200′ for a wound therapy device is illustrated according to another alternate exemplary embodiment. The shell 200′ can be the same or similar to the shell 200 of FIGS. 3-5 in many aspects so like reference numbers are used to illustrate like elements and like reference numbers with the prime symbol are used to illustrate similar elements. For example, the shell 200′ can include apron 206′ and raised portion 208′. Also, the raised portion 208′ can include at least one peripheral wall (i.e., the peripheral walls defined by the ends 208a′-208d′) and upper wall 208e′. However, the upper wall 208e′ on the shell 200′ can include at least one indicator portion (e.g., dimples 240, 242, 244, 246 and 248 in the illustrated embodiment). In one embodiment, the dimples 240-248 are formed as reduced thickness portions. In the same or another embodiment, the dimples 240-248 are formed of a different material having a difference Shore hardness. In one embodiment, the dimples 240-248 collapses when the negative pressure condition exceeds a predetermined negative pressure (e.g., a specific threshold pressure in the range of 50-150 mmHg, such as 100 mmHg) to indicate presence of the negative pressure condition.

In the illustrated embodiment, the at least one reduced thickness portion includes a plurality of dimples 240, 242, 244, 246 and 248. These dimples can be normally (i.e., when no negative pressure condition exists) provided in a convex state, as shown for dimples 240-244. Upon reaching the specific threshold pressure (e.g., 100 mmHg), the dimples can move to the concave state, as shown for dimples 246 and 248, to provide a visual indication that the specific threshold pressure has been reached. The dimples can be provided as reduced thickness portions relative to the surrounding portions of the upper wall 208e′ so that these reduced thickness portions are forcibly moved under the negative pressure condition without the same occurring to the surrounding portions of the upper wall 208e′. Of course, less than five or more than five dimples could be used. Moreover, other reduced thickness configurations can be used (i.e., something other than dimples), including for example indicia corresponding to a trade name or mark. In the same or other embodiments the dimples 240-248 can function as a pressure management feature whereby the dimples 240-248 collapse to reduce volume with in the interior volume 204 and thereby increase the pressure to limit the amount of the negative pressure condition applied within the shell 200′.

In another embodiment, the reduced thickness portion can include a plurality of progressively reduced thickness areas that progressively indicate when the negative pressure condition progressively exceeds a progression of predetermined pressures. For example, the plurality of progressively reduced thickness areas can include the dimples 240-248 with each dimple having a progressively reduced thickness. For example, dimple 240 can have a reduced thickness corresponding to a first progressive threshold pressure (e.g., 50 mmHg) and can deform (i.e., collapse to the concave state) to indicate that the first progressive threshold pressure has been achieved. The dimple 242 can have a reduced thickness corresponding to a second progressive threshold pressure (e.g., 75 mmHg) to indicate that the second progressive threshold pressure has been achieved. Dimples 244-248 can likewise have reduced thicknesses corresponding, respectively, to third, fourth and fifth progressive threshold pressures (e.g., 100 mmHg, 125 mmHg, and 150 mmHg, respectively) to indicate, respectively, that third, fourth and fifth threshold pressures have been achieved. In FIG. 10, dimples 240, 242 and 244 are shown in the concave states indicating that first through third progressive threshold pressures have been exceeded, whereas dimples 246 and 248 are shown in the convex states indicating that the fourth and fifth progressive threshold pressures have not yet been reached. Again, of course, less than five or more than five dimples could be used and/or other reduced thickness configurations can be used as described above.

In still another embodiment, the shell body 202 can include a reduced thickness portion that is collapsible to provide a supplemental negative pressure force with the interior cavity 204 due to resiliency of the reduced thickness portion urging the reduced thickness portion to a pre-collapsed position. For example, the reduced thickness portion could be one or more of the dimples 240-248 with the dimples in a normally convex state. Forcibly, such as via an applied manual force, deforming the dimples 240-248 to the concave state could reduce the volume of the interior cavity 204 slightly. After application of the shell 200 to the skin contacting assembly 28, resiliency of the shell 200 could urge the depressed dimples back toward the convex state thereby providing some supplemental negative pressure force within the interior cavity 204.

Advantageously, the shell body 202 in any of the foregoing embodiments is able to support and maintain a low-pressure air volume while being flexible enough to conform to the contours of the human body in the area of a wound being treated. When used with the pump 100, as the negative pressure condition is created, the developing negative pressure urges the shell body 202 to collapse; however, as described hereinabove, the shell body 202 is sufficiently resistant to such collapse and maintains the interior cavity 204 so that its volume does not decrease more than 19% relative to the volume of the interior cavity 204 prior to the negative pressure condition.

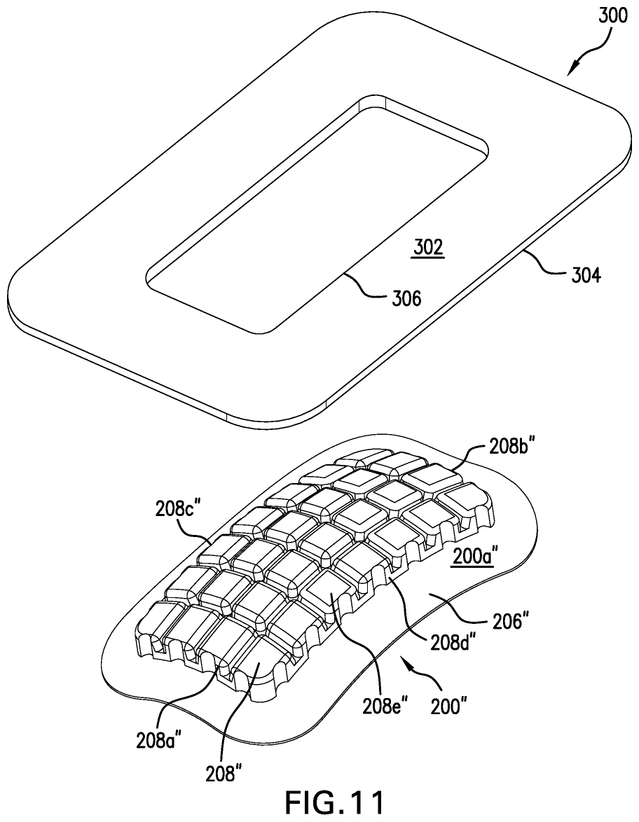

With reference now to FIG. 11, a shell 200″ for a wound therapy device is illustrated according to another alternate exemplary embodiment. The shell 200″ can be the same or similar to the shell 200 of FIGS. 3-5 (or the shell 200′ of FIG. 10) in many aspects so like reference numbers are used to illustrate like elements with a double prime symbol added. For example, the shell 200″ can include apron 206″ and raised portion 208″. Also, the raised portion 208″ can include the at least one peripheral wall (i.e., the peripheral walls defined by the ends 208a′-208d″) and the upper wall 208e″. The apron 206″ can have an enlarged size or footprint outside the raised portion 208″ relative to the aprons of the pump shells 200 or 200′. As will be described in more detail below, this enables the apron 206″ to be secured to the skin S at a location outside that of any skin contacting elements or assemblies.

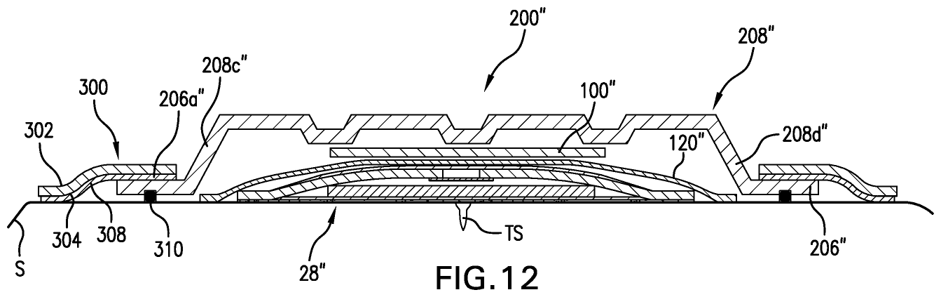

The shell 200″ can also include or be used with a retaining element 300 for overlaying the perimeter apron 206″ and for securing the perimeter apron 206″ to the skin S. More particularly, the retaining element 300 can include an upper side 302 and a lower side 304 with the lower side 304 adherable to the upper side 206″ of the perimeter apron 206″ and adherable to the skin S. The retaining element 300 also includes a central aperture 306 sized to complementarily receive the raised portion 208″. The retaining ring 300 can be formed of a flexible material, such as an elastomeric material that can be a thin, flexible elastomeric film. The lower side 304 of the retaining element includes an adhesive 308 (FIG. 12), which can optionally be spray coated and covered with a release liner (not shown).

With additional reference to FIG. 12, advantageously, an underside 206b″ of the perimeter apron 206″ can be directly sealed via an sealing element 310 to the skin S. This can eliminate the need for any seal between the shell 200″ and a skin contacting assembly or element and can additionally eliminate the need for any seal between any skin contacting assembly or element used beneath the shell 200″ and the skin S. Also advantageously, no adhesive need be applied to the underside 206b″ of the apron 206″. In one embodiment, the shell 200″ with the retaining ring 300 can be used with a skin contacting element or assembly that is smaller in size than an inner dimension of the perimeter apron 206″ so that no contact occurs between the shell 200″ and the skin contacting element or assembly. In the same or another embodiment, the shell 200″ can be provided to an end user with the retaining ring 300 already adhered to the apron 206″ and only needing further adherence to the skin S.

As shown in FIG. 12, a skin contacting assembly 28″ is disposed below the shell 200″, which can be the same as the skin contacting assembly 28 described hereinabove (or any related embodiments discussed herein), though it may be smaller in size to more easily fit within the inner dimensions of the apron 206″. Accordingly, the skin contacting assembly 28″ can be adhered to the skin S as already described herein, though a notable exception is that no sealing element (e.g., sealing element 60) need be used between the skin contacting assembly 28″ and the skin S. Instead, since the perimeter apron 206″ directly contacts the skin S, only the sealing element 310 need be used between the apron 206″ and the skin S. Optionally, pump 100″ can be retained inside the shell 200″ via an air permeable liquid impervious membrane 120″, which can be the same or similar to the membranes 78 or 120.

The retaining element 300 includes the adhesive 308 on the underside 304 thereof. In particular, the underside 304 has an inner portion that overlaps and rests against the upper side 206a″ to adhere thereto and the underside 304 has an outer portion that overlaps and rests against the skin S to adhere thereto. This secures the shell 200″ relative to the skin S and thus to the tissue site TS. In the illustrated embodiment of FIG. 12, the skin contacting assembly 28″ is sized so as to be spaced peripherally relative to the shell 200″, though this is not required and it is contemplated that the apron 206″ could at least slightly overlap the skin contacting assembly 28″.

In the illustrated embodiment, the walls or sides (only sides 208c″ and 208d″ shown) have a uniform thickness with the apron 206″. In alternate embodiments, the apron 206″ could have a reduced thickness relative to the wall so as to reduce the likelihood of any lifting of the apron 206″ after the negative pressure condition is created with the shell 200″.

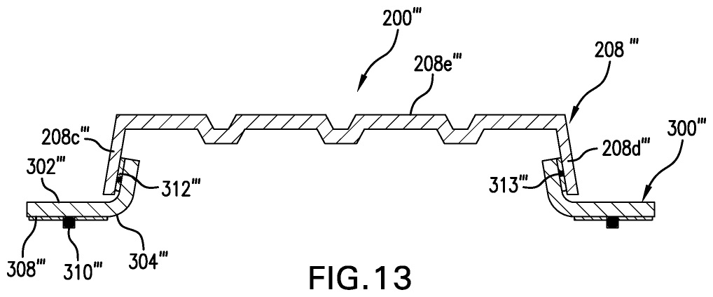

With reference to FIG. 13, a shell 200″ for a wound therapy device is illustrated according to yet another alternate exemplary embodiment. The shell 200″ can be the same or similar to the shell 200″ of FIGS. 11-12 in many aspects so like reference numbers are used to illustrate like elements with a triple prime symbol added. Unlike the shell 200″, the shell 200′″ does not include an integrally formed or unitary apron. Instead, the shell 200′″ includes only the raised portion 208′″ having walls or sides (only walls 208c′″ and 208d′″ shown) depending from upper wall 208e′″ and terminating distally relative to the upper wall 208e″.

The shell 200′″ can also include or be used with a retaining element 300′″ for sealing and securing (or affixing) the shell 200′″ to the skin (e.g., skin S in FIGS. 11-12). In this regard, the retaining element 300′″ can include an upper side 302′″ and a lower side 304′″. The retaining ring 300′″ can be formed of a flexible material, such as an elastomeric material that can be a thin, flexible elastomeric film. The upper side 302′″ of the retaining element 300′″ can be secured to the raised portion 208′″ via an adhesive 312′″ interposed between the retaining element 300′″ and an interior of the raised portion 208′″ (i.e., interior faces of the walls, e.g., walls 208c′″ and 208d′″, of the raised portion 208′″). A seal 313′″ can also be interposed between the retaining element 300′″ and the interior of the raised portion 208′″ for sealing therebetween. The lower side 304′″ of the retaining ring 300′″ can be both sealed and adhered to a wearer's skin. For example, the retaining ring 300′″ can be sealed to the skin via a sealing element 310′″ and can be adhered to the skin via an adhesive 308′″, which can be the same or similar, respectively, to the sealing element 310 (or the pump gasket 108) and the adhesive 308 (or the adhesive 110).

In any of the foregoing pump shell embodiments, the pump 100 can be provided therein and can be as discussed hereinabove regarding the pump 100. For example, the pump 100 can be a chemical pump that creates the negative pressure condition. In particular, the pump 100 can be a chemical pump that scavenges oxygen to create the negative pressure condition, a VOD device, . Alternatively, the pump 100 can be a VOD (vacuum on demand) device, etc. Optionally, the pump shell of any of the foregoing embodiments can include a sealing layer that when removed exposes and activates the pump 100. This could include a sealing layer (or secondary sealing layer) that when removes activates a second pump that provides heat at the tissue site TS. Optionally, the pump body can accommodate a low voltage power supply to enable sensors, LED, LCD, OLED or other displays and/or visual indicators. Also optionally, the pump body can contain a low voltage power supply to provide light therapy and electro stimulation in combination with negative pressure therapy. In one embodiment, these power supplies can be printed, zinc-air, small batteries, etc.

A method for applying negative pressure to a tissue site will be described with reference to the wound therapy device 26, particularly when the shell 200 or 200′ is used in the wound therapy device 26, including the various alternate embodiments regarding the shell 200 or 200′. It is to be appreciated that the method could be accomplished using a wound therapy device that is structurally different than any of the wound therapy devices disclosed herein and thus the method is not intended to be limited to such wound therapy devices.

The method includes covering the tissue site TS with the skin contacting element or assembly 28. This can include removing the release liner 70 to expose the skin contacting side 50 and thus the adhesive 72 and the sealing element 60 when already present on the skin contacting side 50 (or one or both the adhesive 72 and the sealing element 60 can be added after the skin contacting side 50 is exposed. Optionally, an adhesive (such as the adhesive used for adhesive 72) or the material used as a sealing element could be used to stabilize any sutures at the tissue site TS. Covering the tissue site TS with the skin contacting element or assembly 28 can further include removing the peel away layer 38 from the drape 36 to expose the opening 40 to ambient.

The method also includes affixing the shell 200 having the pump (e.g., pump 100) accommodated therein to one or both the skin S surrounding the tissue site TS or the skin contacting element or assembly 28, and includes actuating the pump to create a pressure condition at the tissue site TS. Often, actuating of the pump will occur before the shell 200 is affixed to the skin S and/or the skin contacting element or assembly 28, particularly when the pump is a reactor or zinc/air cell configured to react with a selected gas found in air, though this is not required. As already described herein, advantageously, the shell 200 is configured to maintain the interior cavity 204 at the minimum specified volume while the pump creates the negative pressure condition.

When the shell 200 includes a reduced thickness portion (e.g., the dimples 240-248), the method can also include indicating a negative pressure condition and/or a specific negative pressure threshold obtained in the interior cavity 204. This can include progressively indicating a progression of specific negative pressure thresholds being reached when progressive reduced thickness portions are used. Alternatively, the method can include applying manual pressure to the reduced thickness portion(s) (e.g., dimples 240-248) to collapse the reduced thickness potion(s) to a collapsed state for applying supplemental negative pressure when resiliency of the reduced thickness portion urges the reduced thickness portion to a non-collapsed state.

Embodiments of a wound therapy device and methods of treating a wound site have been described above in particularity. Modifications and alterations will occur to those upon reading and understanding the preceding detailed description. The invention, however, is not limited to only the embodiments described above. Instead, the invention is broadly defined by the appended claims and the equivalents thereof. It will be appreciated that various of the above-disclosed and other features and functions, or alternatives or varieties thereof, may be desirably combined into many other different systems or applications. Also that various presently unforeseen or unanticipated alternatives, modifications, variations or improvements therein may be subsequently made by those skilled in the art which are also intended to be encompassed by the following claims.

1. A shell for a wound therapy device, comprising:
a shell body having an interior cavity for accommodating a negative pressure condition, the shell body configured to be affixed to one or both skin surrounding a tissue site or a skin contacting assembly or element to apply the negative pressure condition at a tissue site over which the skin contacting assembly or element is disposed, the shell body also configured to maintain the interior cavity at a minimum specified volume while accommodating the negative pressure condition.

2. The pump shell of claim 1 wherein the negative pressure condition is 50-150 mmHg.

3. The pump shell of claim 1 wherein the shell body is configured to maintain the interior cavity at the minimum specified volume while a negative pressure condition occurs within the shell body for at least 48 hours.

4. The pump shell of claim 1 wherein the shell body is formed of silicone rubber.

5. The pump shell of claim 4 wherein the silicone rubber has a Shore hardness in the range of about 15A to about 90A, and optionally a Shore hardness of around 70A.

6. The pump shell of claim 1 wherein the shell body is unitarily formed of at least two materials having varying Shore hardnesses.

7. The pump shell of claim 1 wherein the shell body includes:
a perimeter apron; and
a raised portion extending upward from the perimeter apron to define the interior cavity.

8. The pump shell of claim 7 wherein the raised portion has a waffle shape.

9. The pump shell of claim 7 wherein the raised portion includes:
at least one longitudinal groove extending longitudinally from a first longitudinal end of the raised portion to a second, opposite longitudinal end of the raised portion; and
at least one lateral groove extending laterally from a first lateral side of the raised portion to a second, opposite lateral side of the raised portion.

10. The pump shell of claim 9 wherein the at least one longitudinal groove and the at least one lateral groove extend downward from the upper wall of the raised portion a first distance that is less than a second distance extending from the upper wall to the perimeter apron.

11. The pump shell of claim 9 wherein the raised portion includes the upper wall and at least one peripheral wall extending from the perimeter apron to the upper wall, wherein the at least one peripheral wall includes spaced apart embossments to increase rigidity of the at least one peripheral wall.

12. The pump shell of claim 11 wherein the at least one longitudinal groove includes at least two longitudinal grooves, the at least one lateral groove includes at least two lateral grooves, and the spaced apart embossments include one embossment positioned between adjacent ones of the at least two longitudinal grooves and the at least two lateral grooves.

13. The pump shell of claim 12 wherein said one embossment extends from the perimeter apron to the upper wall.

14. The pump shell of claim 9 wherein the raised portion includes an upper wall and at least one peripheral wall extending from the perimeter apron to the upper wall, and wherein the upper wall includes at least one reduced thickness portion that collapses when the negative pressure condition exceeds a predetermined negative pressure to indicate presence of the negative pressure condition.

15. The pump shell of claim 14 wherein the reduced thickness portion includes a plurality of progressively reduced thickness areas that progressively indicate when the negative pressure condition progressively exceeds a progression of predetermined negative pressures.

16. The pump shell of claim 7 further including a retaining element overlaying the perimeter apron, the retaining element having an upper side and a lower side with the lower side adhered to an upper side of the perimeter apron and adhered to the skin.

17. The pump shell of claim 16 wherein a seal element is provided between the underside of the perimeter apron and the skin.

18. The pump shell of claim 7 wherein the raised portion includes tapering.

19. The pump shell of claim 1 wherein the shell body includes a reduced thickness portion that collapses upon the negative pressure condition exceeding a predetermined negative pressure to indicate the presence of the negative pressure condition.

20. The pump shell of claim 1 wherein the shell body includes a reduced thickness portion that is collapsible to provide a supplemental negative pressure force within the interior cavity due to resiliency of the reduced thickness portion urging the reduced thickness portion to a pre-collapsed position.

21. The pump shell of claim 1 wherein the shell body includes an air permeable liquid impervious membrane arranged on an exposure side of the pump so as to be interposed between the pump and the skin contacting assembly or element when the shell body is affixed to the skin contacting assembly or element.

22. The pump shell of claim 1 further including a wicking element interposed between the pump and the tissue site such that the negative pressure condition is applied to the tissue site through the wicking element.

23. A method for applying negative pressure to a tissue site, comprising:
covering the tissue site with a skin contacting element or assembly;
affixing a pump shell having a pump accommodated therein to one or both skin surrounding the tissue site or the skin contacting element or assembly; and
actuating the pump to create a negative pressure condition at the tissue site, wherein the pump shell is configured to maintain an interior cavity thereof at a minimum specified volume while the pump creates the negative pressure condition.

24. The method of claim 23 wherein the pump shell includes a reduced thickness portion and the method further includes:
applying manual pressure to the reduced thickness portion to collapse the reduced thickness portion to a collapsed state for applying supplemental negative pressure and maintains sufficient resiliency of the reduced thickness portion to mechanically or pneumatically urge toward a non-collapsed state to create a negative pressure condition when sealed to the tissue site.

25. The method of claim 23 wherein said affixing the pump shell to one or both the skin surrounding the tissue site or the skin contacting element or assembly occurs after said actuating the pump to create the negative pressure condition.

26.-29. (canceled)

30. The pump shell of claim 7 wherein an angle α between the raised portion and the apron is greater than 90 degrees.

31. The pump shell of claim 1 wherein the pump shell includes a compressible portion configured to receive application of manual pressure or pre-compressed pressure to reduce a portion of the pump shell to a collapsed state for applying negative pressure and to maintain sufficient resiliency to mechanically or pneumatically urge and return the compressible portion to a pre-collapsed state to create a negative pressure condition when sealed to the tissue site.

32. The method of claim 23 further including:
applying manual pressure or pre-compressed pressure to reduce a portion of the pump shell to a collapsed state for applying supplemental negative pressure and maintaining sufficient resiliency to the partially compressed portion the shell to mechanically or pneumatically urge and return the portion to a non-collapsed state to create a negative pressure condition when sealed to the tissue site.

33. A pump shell for a wound therapy device, comprising:
a shell body having an interior cavity for accommodating a pump adapted to create a negative pressure condition upon actuation thereof, the shell body configured to be affixed to one or both skin surrounding a tissue site or a skin contacting assembly or element to create the negative pressure condition at a tissue site over which the skin contacting assembly or element is disposed, the shell body also configured to maintain the interior cavity at a minimum specified volume while the pump creates the negative pressure condition.
